(19) United States
(12) Patent Application Publication
(10) Pub. No.: US 2026/0363985 A1
(43) Pub. Date: Nov. 19, 2026

(54) TWO-STEP DYEING METHOD USING NATURAL DYES WITH IMPROVED COLOR INTENSITY

(71) Applicant: HENKEL AG & CO. KGAA, Düsseldorf (DE)

(72) Inventors: Skrollan KLAAS, Hamburg (DE); Nele DALLMANN, Hamburg (DE); Thomas HIPPE, Appen (DE); Fabian STRASKE, Hamburg (DE); Jana Marie ROESEKE, Tornesch (DE)

(21) Appl. No.: 19/128,589

(22) PCT Filed: Oct. 24, 2023
(86) PCT No.: PCT/EP2023/079536

§ 371 Date: May 9, 2025

(30) Foreign Application Priority Data
Nov. 9, 2022 (DE) 10 2022 211 856.1

(51) Int. Cl. A61K 8/60; A61K 8/19; A61K 8/66; A61K 8/9789; A61Q 5/10

(52) U.S. Cl. CPC A61K 8/602; A61K 8/19; A61K 8/66; A61K 8/9789; A61Q 5/10; A61K 2800/43; A61K 2800/884

(57) ABSTRACT

A two-step method for dyeing keratin-containing fibers, in particular human hair, includes using a natural dye based on flavones or flavone glycosides in combination with at least one glycosylase (EC 3.2) and subsequent application of an iron(II) salt. Compositions and kits for carrying out the dyeing method are also described.

The invention relates to a two-step method for dyeing keratin-containing fibers, in particular human hair, using a natural dye based on flavones or flavone glycosides in combination with at least one glycosylase (EC 3.2) and subsequent application of an iron(II) salt, and to compositions and kits for carrying out said dyeing method.

The desire to change one's hair color is a great need for many consumers. To satisfy this need, the cosmetic industry provides a diverse range of products. Hair coloring agents that achieve particularly long-lasting coloring with high coverage are usually oxidation dyes. These use oxidizing agents, which can damage the hair structure. Certain cationic direct azo dyes are also capable of producing hair color changes with excellent fastness properties. The azo dyes mentioned are synthetic dyes. However, a growing number of consumers are demanding hair coloring agents and hair coloring methods based upon natural dyes, even though these agents and methods are often inferior to the aforementioned agents and methods in terms of fastness properties, coverage, and color variety.

### PRIOR ART

The patent KR102102671 B1 discloses the industrial production of the antioxidant flavone luteolin from extracts of Chrysanthemum by fermentation with lactic acid bacteria of the genus Lactobacillus, which contain the enzyme beta-glucosidase.

### Object

The object of the present invention was to provide a method for coloring keratin-containing fibers, in particular human hair, using naturally occurring substances. A further object of the present invention was to provide a method for coloring keratin-containing fibers, in particular human hair, using naturally occurring substances with which colorings with good coloring properties are achieved, in particular with high color intensity, good wash-, light-, and/or rubbing fastness, good homogeneity, and a good balancing capacity, i.e., a low selectivity between damaged and undamaged regions along the keratin fibers.

Surprisingly, it was found that, by the combined application of at least one flavone-containing or flavone-glycoside-containing plant part and at least one glycosylase (EC 3.2), which is located outside the plant part, to keratin fibers and a subsequent treatment with the solution of an iron (II) salt, colorations of the keratin fibers with good application properties can be achieved.

A first subject matter of the present invention is a method for the non-oxidative dyeing of keratin fibers, in particular human hair, with natural dyes, which method comprises the following method steps in the specified order:
a) providing a composition (F) containing at least one flavone or flavone glycoside and providing a composition (E) containing at least one glycosylase (EC 3.2),
b) preparing a mixture (M) from the compositions (F) and (E),
c) immediately thereafter, within a time of from zero seconds to 10 minutes, preferably 10 seconds to 5 minutes, particularly preferably 30 to 120 seconds, applying the mixture (M) to the keratin fibers, d) allowing to act for a time of from 30 seconds to 60 minutes, preferably 5 to 45 minutes, particularly preferably 15 to 30 minutes,
e) rinsing the keratin fibers with water,
f) optionally drying the keratin fibers,
g) immediately thereafter, within a time of from zero seconds to 10 minutes, preferably 10 seconds to 5 minutes, particularly preferably 30 to 120 seconds, applying an aqueous composition (FE) containing at least one salt of the divalent iron cation Fe(II) in an amount of 0.001-3.0 wt. wt. %, preferably 0.01-2 wt. %, particularly preferably 0.1-1 wt. %, in each case based upon the weight of the composition (FE), and having a pH in the range of 1.0 to 6.0, preferably 2.5 to 5.5, particularly preferably 3.6 to 5.3, extremely preferably 4.5 to 5.2, in each case measured at 20 °C,
h) allowing to act for a time of from 30 seconds to 60 minutes, preferably 5 to 45 minutes, particularly preferably 15 to 30 minutes,
i) rinsing the keratin fibers with water,
j) optionally drying the keratin fibers.

A further subject matter of the present invention is a kit for the non-oxidative dyeing of keratin fibers, in particular human hair, which kit comprises the following compartments, physically separated from each other: a composition (F) containing at least one flavone or flavone glycoside, a composition (E) containing at least one glycosylase (EC 3.2) and an aqueous composition (FE) containing at least one salt of the divalent iron cation Fe(II) in an amount of 0.001-3.0 wt. %, preferably 0.01-2 wt. %, particularly preferably 0.1-1 wt. %, in each case based upon the weight of the composition (FE), and having a pH in the range of 1.0 to 6.0, preferably 2.5 to 5.5, particularly preferably 3.6 to 5.3, extremely preferably 4.5 to 5.2, in each case measured at 20 °C.

### Source of Flavones and Flavone Glycosides

Flavones and flavone glycosides preferably used according to the invention are present
a) as extracts from plants or parts of plants containing flavones or flavone glycosides;
b) as plant parts containing flavones or flavone glycosides. In this case, the glycosylases separately provided according to the invention do not include those glycosylases that are naturally present in the flavone (glycoside)-containing plant parts used.

A preferred embodiment of the invention is characterized in that the flavone-containing or flavone-glycoside-containing plant part, which is used as such in dried form or the extract thereof is used, is selected from the flowers, leaves, stems, branches, bark, trunk wood, berries, fruits, aggregate nuts, aggregate drupes, stone fruits, apple fruits, peels, seeds, cones or roots of at least one flavone-containing or flavone-glycoside-containing plant.

A further preferred embodiment of the invention is characterized in that the at least one flavone or flavone glycoside is selected from luteolin, apigenin, apiin, rutin, kaempferol, quercetin, salvigenin, myricetin and the glycosides derived from the above-mentioned flavones, as well as mixtures of these substances.

In a further preferred embodiment of the invention, the composition (F) is characterized in that it contains, based upon its weight, at least one flavone or flavone glycoside selected from luteolin, apigenin, apiin, rutin, kaempferol, quercetin, salvigenin, myricetin and the glycosides derived from the above-mentioned flavones, as well as mixtures of these substances in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

In a further preferred embodiment of the invention, the composition (M) is characterized in that it contains, based upon its weight, at least one flavone or flavone glycoside selected from luteolin, apigenin, apiin, rutin, kaempferol, quercetin, salvigenin, myricetin and the glycosides derived from the above-mentioned flavones, as well as mixtures of these substances in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

a) Extracts from Plants or Parts of Plants Containing Flavones or Flavone Glycosides

In an extremely preferred embodiment of the invention, at least one extract from at least one flavone-containing or flavone-glycoside-containing plant part is used.

Plant parts preferred according to the invention from which at least one extract preferred according to the invention can be obtained are the flowers, leaves, stems, branches, bark, trunk wood, berries, fruits, aggregate nuts, aggregate drupes, stone fruits, apple fruits, peels, seeds, cones or roots of at least one flavone-containing or flavone-glycoside-containing plant.

Suitable extractants are water, in particular hot water with a temperature of 45-100 °C, furthermore C₁-C₄ alkanols and C₂-C₄ polyols, in particular ethanol, isopropanol, n-propanol, ethylene glycol, 1,2-propanediol, glycerin, and 1,3-butylene glycol, and mixtures of these extractants, in particular mixtures of water and at least one C₁-C₄ alkanol, mixtures of water and at least one C₂-C₄ polyol, particularly preferably water/ethanol mixtures.

The extract itself can preferably be used in concentrated form, obtained by partial distillation of the extractant after extraction, as a viscous liquid. Other extracts preferred according to the invention are used in powder form, obtained by drying, preferably spray drying, the solvent-containing extract.

A preferred embodiment of the invention is characterized in that the at least one flavone or flavone glycoside used according to the invention comes from at least one extract of a plant selected from Reseda, in particular Reseda luteola and Reseda lutea, Genista tinctoria, Matricaria chamomilla, Chrysanthemum, Petroselinum crispum, Cynara, Citrus sinensis, Daucus carota, Apium, Mentha piperita and Perilla frutescens.

In a particularly preferred embodiment of the invention, the at least one flavone or flavone glycoside used according to the invention comes from a plant selected from Reseda, in particular Reseda luteola and Reseda lutea, Genista tinctoria, Chrysanthemum and Matricaria chamomilla.

In a particularly preferred embodiment of the invention, the at least one flavone or flavone glycoside used according to the invention comes from an extract of the flowers, leaves and stems of Reseda luteola.

In a particularly preferred embodiment of the invention, the at least one flavone or flavone glycoside used according to the invention comes from an aqueous-ethanolic extract of the flowers, leaves and stems of Reseda luteola.

Of course, the compositions (F) according to the invention and preferably used according to the invention can also contain plant parts and/or plant extracts of two or more different plants.

Extracts that are preferably used according to the invention contain, in each case based upon their weight, at least one flavone or flavone glycoside in a total amount of 1 to 100 wt. %, preferably 10 to 85 wt. %, particularly preferably 20 to 80 wt. %, and exceptionally preferably 40 to 75 wt. %. Compositions (F) preferably used according to the invention contain, in each case based upon their weight, at least one flavone or flavone glycoside in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

Compositions (M) preferably used according to the invention contain, in each case based upon their weight, at least one flavone or flavone glycoside in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

Compositions (F) preferably used according to the invention contain, in each case based upon their weight, at least one flavone or flavone glycoside from Reseda luteola in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

Compositions (M) preferably used according to the invention contain, in each case based upon their weight, at least one flavone or flavone glycoside from Reseda luteola in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

Compositions (F) preferably used according to the invention contain, in each case based upon their weight, at least one flavone or flavone glycoside from Reseda lutea in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

Compositions (M) preferably used according to the invention contain, in each case based upon their weight, at least one flavone or flavone glycoside from Reseda lutea in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

Compositions (F) preferably used according to the invention contain, in each case based upon their weight, at least one flavone or flavone glycoside from Chrysanthemum in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

Compositions (M) preferably used according to the invention contain, in each case based upon their weight, at least one flavone or flavone glycoside from Chrysanthemum in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

Compositions (F) preferably used according to the invention contain, in each case based upon their weight, at least one flavone or flavone glycoside from Genista tinctoria in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

Compositions (M) preferably used according to the invention contain, in each case based upon their weight, at least one flavone or flavone glycoside from Genista tinctoria in a total amount of 0.01-5 wt. %, preferably 0.05-2 wt. %, particularly preferably 0.1-1 wt. %, extremely preferably 0.2-0.6 wt. %, further extremely preferably 0.3-0.5 wt. %.

### b) Use of Chopped Plant Parts

In a further preferred embodiment of the invention, the plant parts used are used in dried and crushed form. In an extremely preferred embodiment of the invention, flowers and/or leaves and/or stems are used as the flavone-containing or flavone-glycoside-containing plant part, particularly preferably in dried and crushed form. In a further extremely preferred embodiment of the invention, the flowers and/or leaves and/or stems of Reseda luteola are used as the flavone-containing or flavone-glycoside-containing plant part, particularly preferably in dried and crushed form. In a further extremely preferred embodiment of the invention, the flowers and/or leaves and/or stems of Reseda lutea are used as the flavone-containing or flavone-glycoside-containing plant part, particularly preferably in dried and crushed form. In a further extremely preferred embodiment of the invention, the flowers and/or leaves and/or stems of Chrysanthemum are used as the flavone-containing or flavone-glycoside-containing plant part, particularly preferably in dried and crushed form. In a further extremely preferred embodiment of the invention, the flowers and/or leaves and/or stems of Genista tinctoria are used as the flavone-containing or flavone-glycoside-containing plant part, particularly preferably in dried and crushed form.

In a further extremely preferred embodiment of the invention, the at least one flavone-containing or flavone-glycoside-containing plant part is used in powder form.

In a further preferred embodiment of the invention, the composition (F) is characterized in that it contains, based upon its weight, at least one flavone-containing or flavone-glycoside-containing plant part in a total amount of 0.05-20 wt. %, preferably 0.1-10 wt. %, more preferably 0.5-5 wt. %, particularly preferably 0.7-2 wt. %, extremely preferably 1-1.5 wt. %.

In a further preferred embodiment of the invention, the composition (F) is characterized in that it contains, based upon its weight, at least one flavone-containing or flavone-glycoside-containing plant part selected from the flowers and/or leaves and/or stems of at least one plant selected from Reseda, in particular Reseda luteola or Reseda lutea, Genista tinctoria, Matricaria chamomilla, Chrysanthemum, Petroselinum crispum, Cynara, Citrus sinensis, Daucus carota, Apium, Mentha piperita and Perilla frutescens, in a total amount of 0.05-20 wt. %, preferably 0.1-10 wt. %, more preferably 0.5-5 wt. %, particularly preferably 0.7-2 wt. %, extremely preferably 1-1.5 wt. %.

In a further preferred embodiment of the invention, the composition (M) is characterized in that it contains, based upon its weight, at least one flavone-containing or flavone-glycoside-containing plant part in a total amount of 0.05-20 wt. %, preferably 0.1-10 wt. %, more preferably 0.5-5 wt. %, particularly preferably 0.7-2 wt. %, extremely preferably 1-1.5 wt. %.

In a further preferred embodiment of the invention, the composition (M) is characterized in that it contains, based upon its weight, at least one flavone-containing or flavone-glycoside-containing plant part selected from the flowers and/or leaves and/or stems of at least one plant selected from Reseda, in particular Reseda luteola or Reseda lutea, Genista tinctoria, Matricaria chamomilla, Chrysanthemum, Petroselinum crispum, Cynara, Citrus sinensis, Daucus carota, Apium, Mentha piperita and Perilla frutescens, in a total amount of 0.05-20 wt. %, preferably 0.1-10 wt. %, more preferably 0.5-5 wt. %, particularly preferably 0.7-2 wt. %, extremely preferably 1-1.5 wt. %.

Compositions (F) preferably used according to the invention contain water and further contain a buffer system selected from a mixture of a medium-strong or weak acid with its conjugated or corresponding base (or the respective salt) and a mixture of a medium-strong or weak base with its conjugated or corresponding acid.

According to the invention, preferably suitable corresponding acid-base pairs are those that stabilize the aqueous composition (F) used according to the invention in the pH range of 1.0 to 8.0, preferably 2.5 to 7.0, particularly preferably 3.6 to 6.0, extremely preferably 4.5 to 5.6, in each case measured at 20 °C. Particularly preferred buffer systems according to the invention are selected from
mixtures of citric acid and its salts, in particular the alkali metal citrates, in particular the sodium salts, in particular trisodium citrate,
mixtures of tartaric acid and its salts, in particular alkali metal tartrates, in particular potassium salts, in particular potassium hydrogen tartrate,
mixtures of phthalic acid and its salts, in particular potassium salts, in particular potassium hydrogen phthalate,
mixtures of lactic acid and its salts, in particular lactic acid/sodium lactate mixtures,
mixtures of gluconic acid and its salts, in particular gluconic acid/sodium gluconate mixtures,
mixtures of succinic acid and its salts, in particular sodium salts, in particular sodium hydrogen succinate and disodium succinate, and
mixtures of malic acid and its salts, in particular sodium salts, in particular sodium hydrogen malate and disodium malate.

A buffer system that is particularly preferred according to the invention is formed from citric acid and at least one sodium salt of citric acid; mixtures of citric acid and trisodium citrate are extremely preferred.

Other buffer systems, e.g., acetic acid/sodium acetate, are in principle also suitable according to the invention. However, due to the vinegar smell, such a buffer is not acceptable for the production of a cosmetic market product.

Dyeing methods that are preferred according to the invention are characterized in that the aqueous composition (F) contains, in each case based upon their weight, a buffer system in an amount of 0.5-5 wt. %, preferably 0.8-4.5 wt. %, particularly preferably 1.5-3.5 wt. %, extremely preferably 2.1-3.0 wt. %.

Dyeing methods that are preferred according to the invention are characterized in that the aqueous composition (F) contains as a buffer system, in each case based upon its weight, 0.2-1.5 wt. %, preferably 0.3-1.4 wt. %, particularly preferably 0.5-1.1 wt. %, extremely preferably 0.6-0.9 wt. % citric acid and 0.3-3.5 wt. %, preferably 0.5-3.1 wt. %, particularly preferably 1.0-2.4 wt. %, extremely preferably 1.5-2.1 wt. % trisodium citrate.

Dyeing methods that are preferred according to the invention are characterized in that the aqueous composition (F) contains as a buffer system, in each case based upon its weight, 0.2-1.5 wt. %, preferably 0.3-1.4 wt. %, particularly preferably 0.5-1.1 wt. %, extremely preferably 0.6-0.9 wt. % gluconic acid and 0.3-3.5 wt. %, preferably 0.5-3.1 wt. %, particularly preferably 1.0-2.4 wt. %, extremely preferably 1.5-2.1 wt. % sodium gluconate.

Dyeing methods that are preferred according to the invention are characterized in that the aqueous composition (F) contains as a buffer system, in each case based upon its weight, 0.2-1.5 wt. %, preferably 0.3-1.4 wt. %, particularly preferably 0.5-1.1 wt. %, extremely preferably 0.6-0.9 wt. % lactic acid and 0.3-3.5 wt. %, preferably 0.5-3.1 wt. %, particularly preferably 1.0-2.4 wt. %, extremely preferably 1.5-2.1 wt. % sodium lactate.

Dyeing methods that are preferred according to the invention are characterized in that the aqueous composition (F) contains as a buffer system, in each case based upon its weight, 0.2-1.5 wt. %, preferably 0.3-1.4 wt. %, particularly preferably 0.5-1.1 wt. %, extremely preferably 0.6-0.9 wt. % succinic acid and 0.3-3.5 wt. %, preferably 0.5-3.1 wt. %, particularly preferably 1.0-2.4 wt. %, extremely preferably 1.5-2.1 wt. % disodium succinate.

Dyeing methods that are preferred according to the invention are characterized in that the aqueous composition (F) contains as a buffer system, in each case based upon its weight, 0.2-1.5 wt. %, preferably 0.3-1.4 wt. %, particularly preferably 0.5-1.1 wt. %, extremely preferably 0.6-0.9 wt. % succinic acid and 0.3-3.5 wt. %, preferably 0.5-3.1 wt. %, particularly preferably 1.0-2.4 wt. %, extremely preferably 1.5-2.1 wt. % sodium hydrogen succinate.

Dyeing methods that are preferred according to the invention are characterized in that the aqueous composition (F) contains as a buffer system, in each case based upon its weight, 0.2-1.5 wt. %, preferably 0.3-1.4 wt. %, particularly preferably 0.5-1.1 wt. %, extremely preferably 0.6-0.9 wt. % malic acid and 0.3-3.5 wt. %, preferably 0.5-3.1 wt. %, particularly preferably 1.0-2.4 wt. %, extremely preferably 1.5-2.1 wt. % disodium malate.

Dyeing methods that are preferred according to the invention are characterized in that the aqueous composition (F) contains as a buffer system, in each case based upon its weight, 0.2-1.5 wt. %, preferably 0.3-1.4 wt. %, particularly preferably 0.5-1.1 wt. %, extremely preferably 0.6-0.9 wt. % malic acid and 0.3-3.5 wt. %, preferably 0.5-3.1 wt. %, particularly preferably 1.0-2.4 wt. %, extremely preferably 1.5-2.1 wt. % sodium hydrogen malate.

In compositions (F) that are preferably used according to the invention, the water content is 40-95 wt. %, preferably 50-90 wt. %, particularly preferably 60-85 wt. %, in each case based upon the weight of the composition (F).

### Glycosylases (EC 3.2)

In a preferred embodiment of the invention, the at least one glycosylase (EC 3.2) is selected from at least one glycosidase (EC 3.2.1). Glycosidases (EC 3.2.1) are understood to mean enzymes that hydrolyze O-glycosyl components and S-glycosyl components.

Glycosidases (EC 3.2.1) preferred according to the invention are selected from a group of enzymes generally referred to as cellulases.

The term “cellulase” as used herein refers to enzymes that catalyze the hydrolysis of 1,4-beta-D-glucoside bonds that are present in cellulose (cellobiose) and/or lichenin and/or beta-D-glucans.

Cellulases are often also able to hydrolyze the 1,4 bonds in beta-D-glucans, which also have 1,3 bonds in addition to the 1,4 bonds. Cellulases are able to break down cellulose to beta-glucose. Consequently, cellulases act in particular upon cellulose-containing or cellulose derivative-containing functional groups and catalyze their hydrolysis. The determining factor as to whether an enzyme is a cellulase in the context of the invention is its ability to hydrolyze 1,4-beta-D-glucoside bonds in cellulose.

The term “cellulase activity” is defined here as an enzyme that catalyzes the hydrolysis of 1,4-beta-D-glucoside bonds in beta-1,4-glucan (cellulose). Cellulose activity is measured using a standard method, e.g., as follows: Cellulases release glucose from CMC (carboxymethylcellulose). The samples are incubated under defined reaction conditions (100 mM sodium phosphate buffer pH 7.5, 40 °C, 15 min) with a substrate (1.25 wt. % CMC). The reaction with p-hydroxybenzoic acid hydrazide (PAHBAH) in the presence of bismuth produces a yellow dye that can be determined photometrically at 410 nm. The prerequisite is an alkaline pH during the color reaction. The amount of sugar released corresponding to the coloration is a measure of enzyme activity (Lever, Anal. Biochem., 1972, 47 & 1977, 81).

Cellulases can be divided into three categories:
1. Endoglucanase (EC 3.2.1.4), also called endo-1,4-beta-glucanase, beta-1,4-glucanase, avicelase, beta-1,4-endoglucanhydrolase, endo-1,4-beta-D-glucanohydrolase, carboxymethylcellulase, or celludextrinase;
2. Cellulose-1,4-beta-cellobiosidase (non-reducing end) (EC 3.2.1.91), also called exoglucanase, 1,4-beta-cellobiohydrolase, 4-beta-D-glucan cellobiohydrolase (non-reducing end), avicelase, exo-1,4-beta-D-glucanase, or exocellobiohydrolase, releases cellobiose from the non-reducing ends of the β-D-glucan chains by hydrolysis of the (1->4)-beta-D-glucosidic bonds in cellulose and cellotetraose;
3. beta-glucosidase (EC 3.2.1.21), also called cellobiase, beta-D-glucoside glucohydrolase, amygdalase, or gentobiase, hydrolyzes terminal, non-reducing beta-D-glucosyl groups to release beta-D-glucose.

Suitable cellulases include those of bacterial or fungal origin. Chemically modified or protein-engineered mutants are included. Suitable cellulases are cellulases from the genera Bacillus, Pseudomonas, Humicola, Fusarium, Thielvia, Acremonium, e.g., the fungal cellulases from Humicola insolens, Myceliophthora thermophila, and Fusarium oxysporum, which are disclosed in U.S. Pat. Nos. 4,435,307, 5,648,263, 5,691,178, 5,776,757, and WO 89/09259. Particularly suitable cellulases can be alkaline or neutral cellulases with color care properties. Examples of such cellulases are cellulases that are described in EP495257, EP531372, WO 96/11262, WO 96/29397, and WO 98/08940. Other examples are cellulase variants as described in WO 94/07998, EP531315, EP3212777, EP3502243, EP3653705, EP 3653706, U.S. Pat. Nos. 5,457,046, 5,686,593, 5,763,254, WO 95/24471, WO 98/12307 and WO 99/01544, and WO 2019/122520.

Examples of glycosylases and glycosidases with endo-1,4-glucanase activity (EC 3.2.1.4) are described in WO 2002/099091—for example, those having a sequence of at least 97% identity to the amino acid sequence of positions 1 to 773 of SEQ ID NO:2 of WO 2002/099091. A further example can comprise a GH44-xyloglucanase, for example a xyloglucanase enzyme having a sequence of at least 60% identity to positions 40 to 559 of SEQ ID NO:2 of WO 2001/062903.

Other examples of cellulases suitable according to the invention include the GH45 cellulases described in WO 96/29397 and, in particular, variants thereof having substitution, insertion, and/or deletion at one or more of the positions corresponding to the following positions in SEQ ID NO:8 of WO 2002/099091: 2, 4, 7, 8, 10, 13, 15, 19, 20, 21, 25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113,114, 117, 119, 121,133, 136, 137, 138,139, 140a, 141, 143a, 145, 146, 147, 150e, 150j, 151, 152, 153, 154, 155, 156, 157,158, 159, 160c, 160e, 160k, 161, 162, 164, 165, 168, 170, 171, 172, 173, 175, 176, 178, 181, 183, 184, 185, 186, 188, 191, 192, 195, 196, 200, and/or 20, preferably selected from P19A, G20K, Q44K, N48E, Q119H, or Q146R.

Commercially available cellulases include Celluzyme™, Carezyme™, Carezyme Premium™, Celluclean™ (e.g., Celluclean™ 5000L and Celluclean™ 4000T), Celluclean Classic™, Cellusoft™, Endolase®, Renozyme® and Whitezyme™ (Novozymes A/S), Clazinase™ and Puradax HA™ (Genencor International Inc.), KAC-500(B)™ (Kao Corporation), Revitalenz™ 1000, Revitalenz™ 2000 and Revitalenz™ 3000 (DuPont), as well as Ecostone® and Biotouch® (AB Enzymes).

In a further preferred embodiment of the invention, the at least one glycosidase (EC 3.2.1) is selected from endo-1,4-beta-glucanase (EC 3.2.1.4), beta-glucosidase (EC 3.2.1.21), exo-1,4-beta-glucosidase (EC 3.2.1.74), cellulose-1,4-beta-cellobiosidase (EC 3.2.1.176), exo-1,4-beta-D-glucanase (EC 3.2.1.91), and oligoxyloglucan reducing-end-specific cellobiohydrolase (EC 3.2.1.150), and mixtures of these enzymes.

In a further preferred embodiment of the invention, the at least one glycosidase (EC 3.2.1) is selected from endo-1,4-beta-glucanase (EC 3.2.1.4), beta-glucosidase (EC 3.2.1.21), and exo-1,4-beta-D-glucanase (EC 3.2.1.91), and mixtures of these enzymes.

A particularly preferred embodiment of the invention (dyeing method, dyeing kit) is characterized in that the composition (E) contains an enzyme mixture comprising at least one endo-1,4-beta-glucanase (EC 3.2.1.4), at least one beta-glucosidase (EC 3.2.1.21), and at least one enzyme selected from at least one exo-1,4-beta-D-glucanase (EC 3.2.1.91) and at least one oligoxyloglucan reducing-end-specific cellobiohydrolase (EC 3.2.1.150), and mixtures thereof.

A particularly preferred embodiment of the invention (dyeing method, dyeing kit) is characterized in that the composition (E) contains an enzyme mixture comprising at least one endo-1,4-beta-glucanase (EC 3.2.1.4), at least one beta-glucosidase (EC 3.2.1.21), and at least one exo-1,4-beta-D-glucanase (EC 3.2.1.91).

A further particularly preferred embodiment of the invention (dyeing method, dyeing kit) is characterized in that the composition (E) contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, and—in a total amount of 25-35 wt. %—at least one enzyme selected from at least one exo-1,4-beta-D-glucanase (EC 3.2.1.91) and at least one oligoxyloglucan reducing-end-specific cellobiohydrolase (EC 3.2.1.150), and mixtures thereof, wherein the indicated amounts are relative to the total weight of the enzyme mixture.

A further particularly preferred embodiment of the invention (dyeing method, dyeing kit) is characterized in that the composition (E) contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, and at least one exo-1,4-beta-D-glucanase (EC 3.2.1.91) in a total amount of 25-35 wt. %, wherein the indicated amounts are relative to the total weight of the enzyme mixture.

A further particularly preferred embodiment of the invention (dyeing method, dyeing kit) is characterized in that the composition (E) contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, furthermore—in a total amount of 25-35 wt. %—at least one enzyme selected from at least one exo-1,4-beta-D-glucanase (EC 3.2.1.91) and at least one oligoxyloglucan reducing-end-specific cellobiohydrolase (EC 3.2.1.150), and mixtures thereof, as well as at least one other enzyme with cellulase activity in a total amount up to 100 wt. %, wherein the indicated amounts are relative to the total weight of the enzyme mixture.

A further particularly preferred embodiment of the invention (dyeing method, dyeing kit) is characterized in that the composition (E) contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, and at least one exo-1,4-beta-D-glucanase (EC 3.2.1.91) in a total amount of 25-35 wt. %, as well as at least one other enzyme with cellulase activity in a total amount up to 100 wt. %, wherein the indicated amounts are relative to the total weight of the enzyme mixture.

Compositions (E) that are preferably used according to the invention contain, in each case based upon their weight, at least one glycosylase (EC 3.2) in a total amount of 1-50 wt. %, preferably 5-40 wt. %, more preferably 7-30 wt. %, particularly preferably 10-25 wt. %, extremely preferably 15-19 wt. %, and furthermore 5-99 wt. %, preferably 10-90 wt. %, particularly preferably 20-80 wt. %, extremely preferably 40-70 wt. % water. Compositions (E) that are extremely preferably used according to the invention contain, in addition to water and the at least one glycosylase (EC 3.2), in each case based upon their weight, at least one sugar selected from sucrose and D-glucose and mixtures thereof in a total amount of 1-20 wt. %, preferably 5-15 wt. %.

A further particularly preferred embodiment of the invention (dyeing method, dyeing kit) is characterized in that the weight-related mixing ratio of composition (E) and composition (F) is in the range of 0.5-10 parts by weight (E) to 500 parts by weight (F), preferably from 1 to 5 parts by weight (E) to 500 parts by weight (F). The composition (F) preferably used according to the invention thus undergoes only a very slight dilution by mixing with the composition (E).

A further particularly preferred embodiment of the invention (dyeing method, dyeing kit) is characterized in that the composition (M) contains, based upon its weight, at least one glycosylase (EC 3.2) in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

A further particularly preferred embodiment of the invention (dyeing method, dyeing kit) is characterized in that the composition (M) contains, based upon its weight, at least one glycosidase (EC 3.2.1) in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

A further particularly preferred embodiment of the invention (dyeing method, dyeing kit) is characterized in that the composition (M) contains, based upon its weight, at least one glycosidase (EC 3.2.1) selected from at least one cellulase in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

A further particularly preferred embodiment of the invention (dyeing method, dyeing kit) is characterized in that the composition (M) contains, based upon its weight, at least one glycosidase (EC 3.2.1) selected from endo-1,4-beta-glucanase (EC 3.2.1.4), beta-glucosidase (EC 3.2.1.21), and exo-1,4-beta-D-glucanase (EC 3.2.1.91) and mixtures of these enzymes in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

A further particularly preferred embodiment of the invention (dyeing method, dyeing kit) is characterized in that the composition (M) contains, based upon its weight, at least one glycosidase (EC 3.2.1) selected from at least one endo-1,4-beta-glucanase (EC 3.2.1.4), at least one beta-glucosidase (EC 3.2.1.21), and at least one enzyme selected from at least one exo-1,4-beta-D-glucanase (EC 3.2.1.91) and at least one oligoxyloglucan reducing-end-specific cellobiohydrolase (EC 3.2.1.150) and mixtures thereof in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

### Incubation Time

In this context, incubation time is understood to mean the time between the addition of the glycosylase solution (E) to the composition (F) containing the flavone or flavone glycoside and the contact of the mixed composition (M) with the keratin fibers to be treated.

The incubation time according to the invention is zero seconds to 10 minutes, preferably 10 seconds to 5 minutes, particularly preferably 30 to 120 seconds.

Another preferred subject matter of the present invention is a method for the non-oxidative dyeing of keratin fibers, in particular human hair, with natural dyes, which method comprises the following method steps in the specified order:
a) providing a composition (F) containing at least one flavone or flavone glycoside and providing a composition (E) containing at least one glycosylase (EC 3.2),
b) preparing a mixture (M) from the compositions (F) and (E),
c) immediately thereafter, within a time of from zero seconds to 10 minutes, preferably 10 seconds to 5 minutes, particularly preferably 30 to 120 seconds, applying the mixture (M) to the keratin fibers,
d) allowing to act for a time of from 30 seconds to 60 minutes, preferably 5 to 45 minutes, particularly preferably 15 to 30 minutes,
e) rinsing the keratin fibers with water,
f) optionally drying the keratin fibers,
g) immediately thereafter, within a time of from zero seconds to 10 minutes, preferably 10 seconds to 5 minutes, particularly preferably 30 to 120 seconds, applying an aqueous composition (FE) containing at least one salt of the divalent iron cation Fe(II) in an amount of 0.001-3.0 wt. %, preferably 0.01-2 wt. %, particularly preferably 0.1-1 wt. %, in each case based upon the weight of the composition (FE), and having a pH in the range of 1.0 to 6.0, preferably 2.5 to 5.5, particularly preferably 3.6 to 5.3, extremely preferably 4.5 to 5.2, in each case measured at 20 °C,
h) allowing to act for a time of from 30 seconds to 60 minutes, preferably 5 to 45 minutes, particularly preferably 15 to 30 minutes,
i) rinsing the keratin fibers with water, optionally drying the keratin fibers,
   wherein the mixture (M) and/or the aqueous composition (FE) have a temperature of 15-50 °C, preferably 20-40 °C, preferably 25-35 °C and/or wherein the keratin fibers are heated during the entire exposure time of (M) and/or (FE) or at least during part of the exposure time of (M) and/or (FE). Heat is preferably supplied by a heat lamp, a drying hood, or a hair dryer. The heat supplied is within physiologically acceptable limits.

Preferably, the keratin fibers are dried after the compositions (M) and/or (FE) have been rinsed out. Drying can take place without the active supply of heat. However, drying can also be carried out with a supply of heat at a temperature of 25-120 °C, particularly preferably at a temperature of 30-80 °C, extremely preferably at a temperature of 35-60 °C. Heat is preferably supplied by a heat lamp, a drying rod, a drying hood, a straightening iron, or a hair dryer.

The keratin fibers can be dried after each rinsing step using an absorbent cloth, such as a towel. The towel-dried hair can optionally also be dried partially or completely with a hairdryer or other heat source. It is also possible to let the keratin fibers air dry.

pH

Other preferred embodiments of the invention (dyeing method, dyeing kit) are characterized in that the compositions (F), (E), (M) and (FE) used according to the invention have the following pH values, each measured at 20 °C:

Compositions (F) that are preferably used according to the invention have a pH in the range from 1.0 to 8.0, preferably 2.5 to 7.0, particularly preferably 3.6 to 6.0, extremely preferably 4.5 to 5.6.

Compositions (E) that are preferably used according to the invention have a pH in the range from 1.0 to 8.0, preferably 2.5 to 7.0, particularly preferably 3.6 to 6.0, extremely preferably 4.5 to 5.6.

Compositions (M) that are preferably used according to the invention have a pH in the range from 1.0 to 8.0, preferably 2.5 to 7.0, particularly preferably 3.6 to 6.0, extremely preferably 4.5 to 5.6.

Compositions (FE) that are preferably used according to the invention have a pH in the range from 1.0 to 6.0, preferably 2.5 to 5.5, particularly preferably 3.6 to 5.3, extremely preferably 4.5 to 5.2.

The composition (FE) used according to the invention has a pH in the range from 1.0 to 6.0, preferably 2.5 to 5.5, particularly preferably 3.6 to 5.3, extremely preferably 4.5 to 5.2, in each case measured at 20 °C, and contains at least one salt of the divalent iron cation Fe(II) in an amount of 0.001-3.0 wt. %, preferably 0.01-2 wt. %, particularly preferably 0.1-1 wt. %, extremely preferably 0.2-0.5 wt. %, in each case based upon the weight of the composition (FE).

A further essential step of the dyeing method according to the invention is the application of at least one salt of the divalent iron cation Fe(II) in an amount of 0.001-3.0 wt. %, preferably 0.01-2 wt. %, particularly preferably 0.1-1 wt. %, extremely preferably 0.2-0.5 wt. %, in each case based upon the weight of the composition (FE). By sequentially applying the Fe(II) salt used according to the invention, the intensity of the dyeing of the keratin fibers achieved with the at least one flavone or flavone glycoside is surprisingly improved. According to the invention, “divalent” is understood to mean an iron salt whose cationic portion comprises iron with the oxidation state “two.”

According to the invention, preferred anions as anionic counterions for the at least one Fe(II) salt are those that are physiologically compatible. These preferably include halides, particularly preferably chlorides, sulfates and the anions of C1-C6 carboxylic acids, extremely preferably lactic acid, gluconic acid, citric acid, acetic acid, propionic acid, oxalic acid, malonic acid, succinic acid, glutaric acid, galactaric acid (mucous acid), tartaric acid and malic acid, as well as mixtures of these anions.

Preferred dyeing methods according to the invention are therefore characterized in that the composition (FE) contains as an Fe(II) salt at least one compound selected from iron(II) lactate, iron(II) gluconate, iron(II) citrate, iron(II) chloride, iron(II) sulfate, iron(II) acetate, iron(II) propionate, iron(II) oxalate, iron(II) malonate, iron(II) succinate, iron(II) glutarate, iron(II) galactarate, iron(II) tartrate and iron(II) malate, and mixtures of these salts in an amount of 0.001-3.0 wt. %, preferably 0.01-2 wt. %, particularly preferably 0.1-1 wt. %, extremely preferably 0.2-0.5 wt. %, in each case based upon the weight of the composition (FE).

If the composition (FE) contains a plurality of Fe(II) salts, their total amount is 0.001-3.0 wt. %, preferably 0.01-2 wt. %, particularly preferably 0.1-1 wt. %, extremely preferably 0.2-0.5 wt. %, in each case based upon the weight of the composition (FE).

Particularly preferred dyeing methods according to the invention are characterized in that the composition (FE) contains iron(II) lactate as an Fe(II) salt in an amount of 0.001-3.0 wt. %, preferably 0.01-2 wt. %, particularly preferably 0.1-1 wt. %, extremely preferably 0.2-0.5 wt. %, in each case based upon the weight of the composition (FE).

In compositions (FE) preferably used according to the invention, the water content is 40-98 wt. %, preferably 50-97 wt. %, particularly preferably 60-90 wt. %, in each case based upon the weight of the composition (FE).

Optionally, the composition (FE) used according to the invention can contain a buffer system as described above for the composition (F).

Other preferred embodiments of the invention (dyeing methods, dyeing kits) are characterized in that the compositions (F), (E) and (FE) used according to the invention each comprise a cosmetic carrier.

In a first embodiment preferred according to the invention, the carrier is water. Compositions (F), (E) and (FE) that are preferably used according to the invention contain, in each case based upon their weight, at least 40 wt. % water.

In order to optimize the applicability of the composition and its dwell time on the keratin fibers, it is preferred that the compositions (F), (E) and (FE) according to the invention or preferably used according to the invention have a thickened consistency, wherein particularly preferably the compositions (F) and (FE) have a thickened consistency. Because the composition (E) preferably only has a small proportion of the mixture (M), (E) can also be water-thin without affecting the application. Preferably used compositions (F) and (FE) are in the form of gel, cream, or paste. Such carriers ensure a homogeneous distribution and a sufficient dwell time of the composition on the keratin fibers.

The compositions according to the invention and preferably used according to the invention of the kits according to the invention can optionally contain further additives in order to optimize the application properties of this composition. Preferred additives are in particular thickeners, which ensure that the composition stays better on the hair during application.

Compositions particularly preferably used according to the invention contain at least one or more hydrophilic thickeners, which are preferably selected from polysaccharides that are different from cellulose and can be chemically and/or physically modified. Hydrophilic thickeners that are particularly preferred according to the invention are compounds from the group of polysaccharides that are different from cellulose, since the skeletal structures of the polysaccharides are of natural origin and biodegradable. Preferred hydrophilic polysaccharide thickeners are different from celluloses, cellulose ethers, and cellulose esters and are selected from xanthan gum, alginic acids (and their corresponding physiologically acceptable salts, the alginates), agar agar (with the polysaccharide agarose present as the main component in agar agar), starch fractions and starch derivatives such as amylose, amylopectin and dextrins, karaya gum, locust bean gum, gum arabic, pectins, dextrans and guar gum, and mixtures thereof.

In preferred embodiments, the hydrophilic thickener present is xanthan gum, with a view to reliable viscosity adjustment and residue-free application to keratin fibers and the scalp.

Further preferred compositions according to the invention contain, with a view to reliable viscosity adjustment, at least one polymeric thickener selected from
non-ionic polymers such as vinylpyrrolidinone/vinyl acrylate copolymers, polyvinylpyrrolidinone, vinylpyrrolidinone/vinyl acetate copolymers, and polyethylene glycols with a molecular weight of 100,000 to 10,000,000 daltons;
cationic polymers, such as dimethyldiallyl ammonium chloride polymers, acrylamide-dimethyldiallyl ammonium chloride copolymers, dimethylaminoethylmethacrylate vinylpyrrolidinone copolymers quaternized with diethyl sulfate, vinylpyrrolidinone-imidazolinium-methochloride copolymers, and quaternized polyvinyl alcohol;
zwitterionic and amphoteric polymers, such as acrylamidopropyl-trimethylammonium chloride/acrylate copolymers and octylacrylamide/methyl methacrylate/tert-butylaminoethyl methacrylate/2-hydroxypropyl methacrylate copolymers, diallyldimethylammonium chloride/acrylate copolymers, t-butylaminoethyl methacrylate/N-(1,1,3,3-tetramethylbutyl)acrylamide/acrylate(/methacrylate) copolymers;
anionic homo- and copolymers based upon polyacrylic acids, polymethacrylic acids, cross-linked polyacrylic acids, cross-linked polymethacrylic acids, the esters and amides of possibly cross-linked poly(meth)acrylic acids, homo- and copolymers of acrylamido-2-methylpropanesulfonic acids, vinyl acetate/crotonic acid copolymers, vinylpyrrolidinone/vinyl acrylate copolymers, vinyl acetate/butyl maleate/isobornyl acrylate copolymers, methyl vinyl ether/maleic anhydride copolymers, and acrylic acid/ethyl acrylate/N-t-butyl acrylamide terpolymers, each in their acid or salt form.

Compositions that are particularly preferably used according to the invention contain, in each case based upon their weight, at least one hydrophilic thickener in a total amount of 0.1 to 5 wt. %, preferably of 0.5 to 4 wt. %, more preferably of 1 to 3.5 wt. %, and very particularly preferably of 1.2 to 2 wt. %.

Compositions particularly preferably used according to the invention contain at least one organic solvent that has a phenyl group in the molecule. Preferably, this solvent is selected from phenoxyethanol, benzyl alcohol, and mixtures thereof. Surprisingly, it was found that such aromatic solvents can have a positive effect on the coloring results of the coloring method according to the invention.

In a further preferred embodiment of the present invention, the compositions preferred according to the invention contain, in each case based upon their weight, 0.1 to 3 wt. %, preferably 0.5 to 2.5 wt. %, more preferably 0.8 to 1.0 wt. %, of at least one organic solvent that has a phenyl group in the molecule. In a further preferred embodiment of the present invention, the compositions according to the invention contain, in each case based upon their weight, 0.1 to 3 wt. %, preferably 0.5 to 2.5 wt. %, more preferably 0.8 to 1.0 wt. %, of at least one organic solvent selected from phenoxyethanol, benzyl alcohol and mixtures thereof.

Further compositions that are particularly preferably used according to the invention contain at least one aliphatic solvent selected from C₁-C₄ alkanols and C₂-C₁₂ polyols, in particular selected from ethanol, isopropanol, n-propanol, ethylene glycol, 1,2-propanediol, glycerin, diethylene glycol, dipropylene glycol, 1,3-butylene glycol, 1,2-hexanediol, 1,6-hexanediol, and 1,2-octanediol, and mixtures of these solvents.

Further compositions that are particularly preferably used according to the invention contain, based upon their weight, at least one aliphatic solvent selected from C₁-C₄ alkanols and C₂-C₁₂ polyols, in a total amount of 0.01-60 wt. %, preferably 0.1-30 wt. %, particularly preferably 0.5-20 wt. %, extremely preferably 1-10 wt. %, further preferably 2-5 wt. %.

Other compositions that are particularly preferably used according to the invention are characterized in that they do not contain an aliphatic solvent selected from C₁-C₄ alkanols and C₂-C₁₂ polyols.

Compositions particularly preferably used according to the invention contain at least one oil. Preferred cosmetic oils are selected from natural and synthetic hydrocarbons, particularly preferably from paraffin oils, C₁₈-C₃₀ isoparaffins, in particular isoeicosane, polyisobutenes and polydecenes, C₈-C₁₆ isoparaffins, and 1,3-di-(2-ethylhexyl)cyclohexane; the benzoic acid esters of linear or branched C₈-22 alkanols; fatty alcohols having 6-30 carbon atoms that are unsaturated or branched and saturated or branched and unsaturated; triglycerides of linear or branched, saturated or unsaturated, optionally hydroxylated C-30 fatty acids, in particular natural oils; the dicarboxylic acid esters of linear or branched C₂-C₁₀ alkanols; the esters of the linear or branched, saturated or unsaturated fatty alcohols having 2-30 carbon atoms with linear or branched, saturated or unsaturated fatty acids having 2-30 carbon atoms, which can be hydroxylated; the addition products of 1 to 5 propylene oxide units to mono- or polyvalent C₈₋₂₂ alkanols; the addition products of at least 6 ethylene oxide units and/or propylene oxide units to mono- or polyvalent C₃₋₂₂ alkanols; the C₈-C₂₂ fatty alcohol esters of monovalent or polyvalent C₂-C₇ hydroxycarboxylic acids; the symmetrical, unsymmetrical, or cyclic esters of carbonic acid with C₃₋₂₂ alkanols, C₃₋₂₂ alkanediols or C₃₋₂₂ alkanetriols; the esters of dimers of unsaturated C₁₂-C₂₂ fatty acids (dimer fatty acids) with monovalent linear, branched, or cyclic C₂-C₁₈ alkanols or with polyvalent linear or branched C₂-C₆ alkanols; silicone oils, and mixtures of the aforementioned substances.

Other compositions that are particularly preferably used according to the invention contain at least one surfactant or emulsifier.

Surfactants and emulsifiers in the sense of the present application are amphiphilic (bifunctional) compounds that consist of at least one hydrophobic and at least one hydrophilic molecule part. The hydrophobic group is preferably a hydrocarbon chain having 8-28 carbon atoms, which can be saturated or unsaturated, linear or branched. This C₈-C₂₈ alkyl chain is particularly preferably linear. The basic properties of the surfactants and emulsifiers are oriented absorption at boundary surfaces and also the aggregation to micelles and the formation of lyotropic phases. When selecting surfactants suitable according to the invention, it may be preferable to use a mixture of surfactants in order to optimally adjust the stability of the compositions according to the invention. Preferred surfactants and emulsifiers are selected from anionic, cationic, zwitterionic, amphoteric, and nonionic surfactants and emulsifiers and mixtures of these substances.

Other compositions that are particularly preferred according to the invention contain at least one linear saturated alkanol having 12-30 carbon atoms.

Preferred linear saturated alkanols having 12-30 carbon atoms, in particular having 16-22 carbon atoms, are selected from cetyl alcohol, stearyl alcohol, arachidyl alcohol, behenyl alcohol, and lanolin alcohol, and mixtures of these alkanols. Alkanol mixtures that are particularly preferred according to the invention are those obtained from the industrial hydrogenation of vegetable and animal fatty acids. Preferably, the total amount of at least one linear saturated alkanol having 12-30 carbon atoms is 0.1-20 wt. %, preferably 0.5-16.5 wt. %, and particularly preferably 3-10 wt. %, in each case based upon the weight of the composition according to the invention.

Non-oxidative dyeing methods and dyeing kits that are preferred according to the invention are further characterized in that no oxidizing agents other than atmospheric oxygen are used therein. In particular, the oxidizing agents hydrogen peroxide, persulfates, perbromates, percarbonates, perborates and percarbamides are excluded. Atmospheric oxygen is not an oxidizing agent in the context of the invention.

In order to make the compositions used according to the invention also olfactorily attractive for the user, further compositions particularly preferably used according to the invention are characterized in that they contain at least one fragrance compound or odoriferous compound.

Compositions that are extremely preferably used according to the invention are characterized in that they contain, based upon their weight, at least one fragrance in a total amount of 0.01-5 wt. %, preferably 0.1-3 wt. %, particularly preferably 0.5-2 wt. %, extremely preferably 1-1.5 wt. %.

What has been said regarding preferred embodiments of the dyeing method according to the invention applies mutatis mutandis to preferred embodiments of the dyeing kits according to the invention and their individual components, the compositions (F), (E) and (FE).

### EMBODIMENTS

The exemplary embodiments presented below are intended to explain the subject matter of the invention in more detail, without limiting it thereto.

The coloring method according to the invention was carried out on strands of white buffalo belly hair (tied in a round shape, approx. 8 cm of free hair).

The test dye used for all dyeing experiments described below was a 1 wt. % solution of the raw material “Weld Extract C08” from Croda, a powdered extract of flowers, leaves and/or stems of Reseda luteola, in water, adjusted to a pH of 5.6, measured at 20 °C, with a sodium citrate buffer (composition (F)).

The raw material used was “Weld Extract C08” from Croda. According to the manufacturer, it is an aqueous-ethanolic extract of flowers, leaves and/or stems of Reseda luteola that has been spray dried and, in order to improve storage stability, contains 15-35 wt. % maltodextrin, based upon the weight of the raw material “Weld Extract C08” used.

A glycosylase mixture (EC 3.2) obtained from Novozymes, hereinafter referred to as “cellulase blend,” was used as composition (E). This enzyme mixture comprised at least one endo-1,4-beta-glucanase (EC 3.2.1.4) in a total amount of 20-35 wt. %, at least one beta-glucosidase (EC 3.2.1.21) in a total amount of 15-25 wt. %, furthermore at least one enzyme selected from at least one exo-1,4-beta-D-glucanase (EC 3.2.1.91) and at least one oligoxyloglucan reducing-end-specific cellobiohydrolase (EC 3.2.1.150) and mixtures thereof in a total amount of 25-35 wt. %, as well as at least one other enzyme with glycosylase activity in a total amount up to 100 wt. %, wherein the indicated amounts are relative to the total weight of the enzyme mixture.

The enzyme active substance content in the mixture used, which was present as an aqueous composition (E), was 190 μg/μL, i.e., 19 wt. %. Furthermore, the “cellulase blend” contained, based upon its weight, 66 wt. % water and 15 wt. % of a sucrose/D-glucose mixture.

After stirring the composition (F) for 2 minutes, 100 μl of cellulase blend corresponding to a composition (E) used according to the invention were added. This mixture, corresponding to a composition (M) used according to the invention, was stirred for a further 2 minutes.

Buffalo belly hair strands were immersed in the composition (M) for 30 minutes while stirring. The liquor ratio (amount of composition (M) per gram of hair) was 50 ml of composition (M) per gram of hair.

Unless otherwise stated, hair treatment solutions (F), (E), (M) and (FE) were at a temperature of 20 °C (room temperature).

After the treatment with the composition (M), the hair strands were rinsed under running deionized water for 30 seconds and combed 20 times (20 °C). The hair strands were then dried with a commercially available hair dryer at a defined distance (d=10 cm) and a defined temperature (T=80±5 °C) while combing them 20 times. The strands were then colorimetrically measured for the first time.

After treatment with the mixture Reseda luteola/Cellulase no color change was observed on the hair fiber.

The following treatment was carried out with Reseda-luteola-/Cellulase-treated strands for a further 30 minutes in 50 ml of an aqueous solution of 1 wt. % iron(II) lactate (Fe_{Lac}), which had a pH value of 5.2 (20 °C), see Table 1 (liquor ratio: 50 ml metal salt solution to 1 g hair strand). After the exposure time had elapsed, the strand was washed out again and dried (see above). The color impression can be seen immediately.

TABLE 1
Composition used according to the invention (FE)

| | |
|---|---|
| Iron(II) lactate (anhydrous) | 1.00 |
| Water, demineralized | 99.00 |
| Total | 100.00 |
| pH value (20 °C) | 5.2 |

Compared to the control (same dyeing procedure/conditions, without the addition of cellulase), the strand dyed with cellulase appears darker. They differ according to CIELAB color space by a ΔE of 10.94 (see Table 2).

Coloring with Reseda luteola without Enzyme Addition

For control purposes, hair strands, not according to the invention, were dyed only with “Weld Extract C08” without enzyme addition. The colorimetric values are listed in Table 2.

TABLE 2
CIELAB measurements with Reseda-luteola- and Fe(II)-treated
strands (control: without cellulase) with mean values and a
ΔE value from the control strands to the untreated strand
and a ΔE value from the mean value of the Reseda-luteola-/
Cellulase/Fe(II)-treated strands to the mean of the control strands

| Batch Name | CIE L | CIE a | CIE b | ΔE |
|---|---|---|---|---|
| Untreated | 77.04 | −0.43 | 7.94 | to the untreated |
| Control1 | 46.95 | 1.19 | 16.18 | hair strand |
| Control2 | 47.68 | 0.72 | 16.01 | |
| Control3 | 46.86 | 1.05 | 16.05 | |
| Average | 47.16 | 0.99 | 16.08 | 40 |
| CellulaseMeta1 | 37.15 | 0.53 | 14.29 | |
| CellulaseMeta2 | 36.51 | 0.78 | 14.48 | to the MV of the |
| CellulaseMeta3 | 35.44 | 0.77 | 14.25 | control |
| Average | 36.37 | 0.69 | 14.34 | 10.94 |

Treating hair strands with Reseda (Reseda luteola flower/leaf/stem extract) in combination with a cellulase blend and subsequent treatment with iron(II) lactate led to an intensified color result.

### Determination of the Hue Achieved by the Method According to the Invention

All colorimetric measurements were carried out using the Spectraflash SF 600 colorimetric device from Datacolor.

The color difference, also known as dE or ΔE, can be easily determined colorimetrically using a colorimeter that measures colors in the L*, a*, b* color space—for example, a Datacolor colorimeter, type Spectraflash SF 600.

The L*, a*, b* color space refers to the CIELAB color space. The L value represents the brightness of the color (black-white axis) and the intensity of the color: the larger the L value, the brighter the color; the smaller the L value, the more intense the color. The a value represents the red-green axis of the system; the greater this value, the more the color is shifted towards red. The b value represents the yellow-blue axis of the system; the greater this value, the more the color is shifted towards yellow.

The color shift ΔE, i.e., the color difference between two (hair) colors, for each of which an L*, a*, b* value combination was determined, is calculated according to the following formula:

ΔE=(ΔL²+Δa²+Δb²)^{0.5}

The greater the value for ΔE, the more pronounced the color difference.

For the spectrophotometer measurements, a D65 lamp and a diffuse/8° optical configuration were used. The spectral reflectance data for each sample from 380 nm to 700 nm were converted to colorimetric data using DCI Color software. Reflectance measurements were determined for each hair sample, wherein the average of 4 measurements was recorded.

The color difference (ΔE) between uncolored strand and colored strand was calculated according to the following formula:

ΔE=)(Lv−Ln)²+(av−an)²+(bv−bn)², where
Lv, av, bv: colorimetric values for colored strand

1. A method for non-oxidative dyeing of keratin fibers, the method comprising:
providing a composition comprising at least one flavone or flavone glycoside;
providing a composition comprising at least one glycosylase;
preparing a mixture of the composition comprising at least one flavone or flavone glycoside and the composition comprising at least one glycosylase;
applying the mixture to the keratin fibers;
maintaining the mixture in the keratin fibers for a time of from 30 seconds to 60 minutes;
rinsing the keratin fibers for a first time;
applying an aqueous composition comprising at least one salt of the divalent iron cation Fe(II) in an amount of from 0.001-3.0 wt. %, based on the total weight of the aqueous composition, and having a pH of from 1.0 to 6.0 measured at 20 °C;
maintaining the aqueous composition in the keratin fibers for a time of from 30 seconds to 60 minutes; and
rinsing the keratin fibers for a second time.

2. The method of claim 1, wherein the at least one glycosylase is at least one glycosidase.

3. The method of claim 2, wherein the at least one glycosidase is at least one cellulase.

4. The method of claim 2, wherein the at least one glycosidase is selected from the group consisting of endo-1,4-beta-glucanase, beta-glucosidase, exo-1,4-beta-glucosidase, cellulose-1,4-beta-cellobiosidase, exo-1,4-beta-D-glucanase, oligoxyloglucan reducing-end-specific cellobiohydrolase, and any mixture thereof.

5. The method of claim 1, wherein the composition comprising at least one glycosylase comprises an enzyme mixture comprising:
at least one endo-1,4-beta-glucanase in an amount of from 20-35 wt. %, based on the total weight of the enzyme mixture,
at least one beta-glucosidase in an amount of from 15-25 wt. %, based on the total weight of the enzyme mixture,
at least one exo-1,4-beta-D-glucanase in an amount of from 25-35 wt. %, based on the total weight of the enzyme mixture, and
at least one other enzyme with cellulase activity,
wherein the at least one endo-1,4-beta-glucanase, the at least one beta-glucosidase, the at least one exo-1,4-beta-D-glucanase, and the at least one other enzyme are together in an amount of 100 wt. %, based on the total weight of the enzyme mixture.

6. The method of claim 1, wherein the mixture comprises the at least one glycosylase in an amount of from 0.0001-1 wt. %, based on the total weight of the mixture.

7. The method of claim 1, wherein the composition comprising at least one glycosylase comprises, based on the total weight of the composition comprising at least one glycosylase:
the at least one glycosylase in an amount of from 1-50 wt. %, and
water in an amount of from 5-99 wt. %.

8. The method of claim 1, wherein the at least one flavone or flavone glycoside originates from at least one extract of a plant selected from the group consisting of Reseda, Genista tinctoria, Matricaria chamomilla, Chrysanthemum, Petroselinum crispum, Cynara, Citrus sinensis, Daucus carota, Apium, Menthapiperita, and Perilla frutescens.

9. The method of claim 8, wherein the at least one extract of the plant is extracted with an extractant selected from the group consisting of water, C₁-C₄ alkanols, C₂-C₄ polyols, and any mixture thereof.

10. The method of claim 1, wherein the at least one flavone or flavone glycoside is selected from the group consisting of luteolin, apigenin, apiin, rutin, kaempferol, quercetin, salvigenin, myricetin, glycosides derived thereof, and any mixture thereof.

11. The method of claim 1, wherein the composition comprising at least one flavone or flavone glycoside comprises the at least one flavone or flavone glycoside in an amount of from 0.01-5 wt. %, based on the total weight of the composition comprising at least one flavone or flavone glycoside.

12. The method of claim 1, wherein the mixture comprises the at least one flavone or flavone glycoside in an amount of from 0.01-5 wt. %, based on the total weight of the mixture.

13. The method of claim 1, wherein the mixture has a pH of from 1.0 to 8.0 measured at 20 °C.

14. A kit for non-oxidative dyeing of keratin fibers, the kit comprising:
a composition comprising at least one flavone or flavone glycoside;
a composition comprising at least one glycosylase; and
an aqueous composition comprising at least one salt of the divalent iron cation Fe(II) in an amount of from 0.001-3.0 wt. %, based on the total weight of the aqueous composition, and having a pH of from 1.0 to 6.0 measured at 20 °C.

15. (canceled)

16. The kit of claim 14, wherein the composition comprising at least one flavone or flavone glycoside, the composition comprising at least one glycosylase, and the aqueous composition are separately packaged.

17. The kit of claim 14, wherein the keratin fibers are human hair.

18. The method of claim 1, wherein the keratin fibers are human hair.

19. The method of claim 1, further comprising drying the keratin fibers after rinsing the keratin fibers for the first time.

20. The method of claim 1, further comprising drying the keratin fibers after rinsing the keratin fibers for the second time.

21. The method of claim 1, wherein the pH of the aqueous composition is of from 4.5 to 5.2 measured at 20 °C.
